(19) United States
(12) Patent Application Publication
(10) Pub. No.: US 2025/0090445 A1
(43) Pub. Date: Mar. 20, 2025

(54) HYALURONIC ACID COMPOSITION

(71) Applicant: Louna Aesthetics SAS, Poisy (FR)

(72) Inventor: Farid HADJAB, Epagny (FR)

(73) Assignee: Louna Aesthetics SAS, Poisy (FR)

(21) Appl. No.: 18/965,615

(22) Filed: Dec. 2, 2024

Related U.S. Application Data
(63) Continuation of application No. PCT/EP2023/064226, filed on May 26, 2023.

(30) Foreign Application Priority Data
May 30, 2022 (DE) 10 2022 113 526.8

(51) Int. Cl. A61K 8/73; A61K 8/19; A61K 8/44; A61K 8/67; A61K 31/728; A61K 47/18; A61K 47/22; A61Q 19/00

(52) U.S. Cl. CPC A61K 8/735; A61K 8/19; A61K 8/447; A61K 8/675; A61K 31/728; A61K 47/183; A61K 47/22; A61Q 19/00; A61K 2800/10; A61K 2800/52; A61K 2800/522; A61K 2800/546; A61K 2800/91

(57) ABSTRACT

The invention relates to a method for preparing a composition containing hyaluronic acid and/or a salt of hyaluronic acid, as well as at least one biologically active molecule. The method comprises the steps of providing a solution that contains hyaluronic acid and/or a salt of hyaluronic acid, adding an alkaline solution, adding the at least one biologically active molecule, and adding an acid solution to obtain the composition. The invention additionally comprises a composition prepared by the method according to the invention and a use of the stated composition for aesthetic and/or therapeutic use in humans and/or animals.

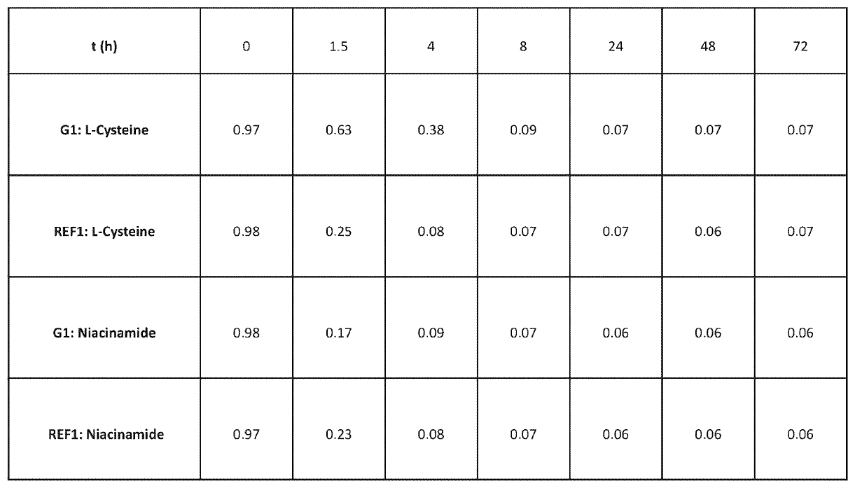

This nonprovisional application is a continuation of International Application No. PCT/EP2023/064226, which was filed on May 26, 2023, and which claims priority to German Patent Application No. 10 2022 113 526.8, which was filed in Germany on May 30, 2022, and which are both herein incorporated by reference.

### BACKGROUND OF THE INVENTION

### Field of the Invention

The invention relates to a method for preparing a composition, a composition prepared in this way, and a use of the composition, wherein the composition contains hyaluronic acid and/or at least one salt of hyaluronic acid, as well as at least one biologically active molecule.

### Description of the Background Art

It is known from the prior art to inject compositions containing hyaluronic acid into human tissue for aesthetic and therapeutic uses. Hyaluronic acid is also referred to in the literature as hyaluronan. Hyaluronic acid is a polysaccharide formed by a plurality of disaccharide repeating units, which in each case comprise D-glucuronic acid and N-acetyl-D-glucosamine. Hyaluronic acid is found in high concentrations in the skin, in the synovia and in the vitreous humor of the eye. A person with a body weight of 70 kg contains approximately 15 g of hyaluronic acid, half of which is present in the tissue. The amount of hyaluronic acid decreases with age.

In many applications it is known to inject compositions containing native and/or chemically modified hyaluronic acid and/or the salts thereof. The disadvantage is that native hyaluronic acid and/or the salts thereof have only a short half-life after injection and are degenerated and broken down comparatively quickly, and therefore the action time of the composition is very limited. To increase the half-life of hyaluronic acid after injection of the composition, the hyaluronic acid and/or the salts thereof are stabilized by cross-linking during the preparation of the composition. However, the cross-linking agents used in this context are toxic, which compromises the safety of a composition containing cross-linked hyaluronic acid and/or the salts thereof.

### SUMMARY OF THE INVENTION

It is therefore an object of the invention to eliminate the aforementioned disadvantages of the prior art and in particular to prepare a composition that is safer to use and nevertheless has the advantages of stabilized hyaluronic acid and/or the salt thereof. The same applies, mutatis mutandis, to the composition and the use thereof.

The object of the invention is achieved by a composition containing hyaluronic acid and/or a salt of hyaluronic acid, as well as at least one biologically active molecule, the method comprising the following steps: providing a solution that contains hyaluronic acid and/or a salt of hyaluronic acid; adding an alkaline solution; adding the at least one biologically active molecule; and adding an acid solution to obtain the composition.

The object of the invention is also achieved by a composition that is prepared by the method according to the invention. Furthermore, the object of the invention is achieved by a use of the composition according to the invention for aesthetic and/or therapeutic use in humans and/or animals.

The invention is based on the basic idea that the preparation method according to the invention eliminates the need for additives stabilizing the hyaluronic acid and/or the salt of hyaluronic acid, for example for cross-linking. The stabilizing effect is achieved in the method according to the invention by the fact that the molecules of hyaluronic acid and/or of the salt of hyaluronic acid are spatially expanded and therefore swell as a result of the addition of the alkaline solution. As a result, the at least one biologically active molecule, which according to the invention is only added after the addition of the alkaline solution, can enter the thus created interstices of the hyaluronic acid and/or of the salt of the hyaluronic acid. The subsequent addition of the acid solution ensures not only that the composition prepared according to the invention has a desired, in particular neutral, pH, but also that the acid solution changes the stereochemistry of the molecules of hyaluronic acid and/or of the salt of hyaluronic acid in such a way that they contract again. As a result, the biologically active molecules in particular are enclosed in the interstices of the molecules of hyaluronic acid and/or of the salt of hyaluronic acid.

This causes a delayed, sustained release of the biologically active molecules after the application of the composition prepared according to the invention, so that its effectiveness is improved, in particular a longer biological activity of the at least one biologically active molecule is made possible. The composition prepared according to the invention thus has the same advantages as a composition having hyaluronic acid and/or salt of hyaluronic acid that has been cross-linked with further additives, but not the disadvantages necessarily associated therewith.

The hyaluronic acid and/or the salt of hyaluronic acid can be obtained from animal sources and/or by bacterial fermentation. The salt of hyaluronic acid used is preferably a physiologically compatible salt of hyaluronic acid, which includes in particular the sodium salt, the calcium salt, the potassium salt, the zinc salt and mixtures thereof. For physiological reasons, the sodium salt of hyaluronic acid is preferably used, which is also called sodium hyaluronate, NaHA or, according to the international nomenclature, hyaluronan sodium. Sodium hyaluronate is stable, soluble and diffusible and therefore physiologically compatible. The concentration of hyaluronic acid and/or the salt of hyaluronic acid can be, preferably after addition of the alkaline solution, between 5 mg/ml and 150 mg/ml, in particular between 30 mg/ml and 130 mg/ml, most preferably between 50 mg/ml and 110 mg/ml.

In a development of the invention, it can be provided for the alkaline solution to contain water and at least one hydroxide component. The water of the alkaline solution can be at least one member of the group of purified water, ultrapure water, ultra-ultrapure water and water for injection. The hydroxide component can contain an alkali metal hydroxide, in particular potassium hydroxide (KOH). Preferably, the hydroxide component is sodium hydroxide (NaOH), in particular at a concentration of 1 N in the alkaline solution.

Examples of the invention can provide for the concentration of the hydroxide component in the alkaline solution to be between 0.05 N and 1.5 N, in particular between 0.1 N and 1 N, preferably between 0.25 N and 1 N. After the alkaline solution is added, a certain time interval can be allowed to pass before the biologically active molecule is added. Within the meaning of the invention, this time interval is also referred to as dissolution time and can be between 5 min and 120 min, in particular between 20 min and 90 min, preferably between 30 min and 60 min. The addition of the alkaline solution can include mixing the composition, wherein the mixing operation can take place during and/or after the addition of the alkaline solution, which contributes to faster homogenization of the composition. Mixing can be carried out at room temperature and/or under pressure and/or using ultrasonic signals to improve homogenization of the composition.

Preferably, the at least one biologically active molecule within the meaning of the invention can cause a biological effect on skin cells, in particular fibroblasts, keratinocytes, melanocytes and/or adipocytes. The at least one biologically active molecule is preferably selected according to the intended use of the composition according to the invention. Within the meaning of the invention, biologically active molecules include comparatively large, in particular natural molecules, such as proteins, in particular cytokines, and lipids, as well as comparatively small molecules that regulate specific signaling pathways and can contribute to cell programming and/or tissue repair. Within the meaning of the invention, the at least one biologically active molecule is preferably cell-permeable, non-immunogenic and can reversibly and selectively modulate intracellular methods of target cells of the biologically active molecule.

Preferably, the at least one biologically active molecule is selected from the group of amino acid, amino ester, hydroxy ester, aldehyde, a derivative of aldehyde, a ketone containing a plurality of hydroxyl groups, a derivative of a ketone containing a plurality of hydroxyl groups, cysteine and/or L-cysteine. The addition of the at least one biologically active molecule can include mixing the composition, wherein the mixing can take place during the addition and/or after the addition of the at least one biologically active molecule. In this way, an improved homogenization of the ingredients of the composition is achieved.

The acid solution can contain hydrochloric acid, in particular concentrated hydrochloric acid. The addition of the acid solution can include mixing the composition, wherein the mixing of the composition can take place during and/or after the addition of the acid solution. In this way, an improved homogenization of the ingredients of the composition is achieved.

According to the invention, the method can provide for the addition of at least one stabilizing agent, wherein the stabilizing agent within the meaning of the invention is preferably designed to protect the molecules of hyaluronic acid and/or of the salt of hyaluronic acid from damaging influences.

The stabilizing agent can comprise at least one antioxidant, in particular an antioxidant vitamin and/or a derivative of an antioxidant vitamin, in order to protect the hyaluronic acid and/or the salt thereof present in the composition from degeneration by free radicals. For example, the stabilizing agent comprises at least one carboxylic acid from the group of antioxidant vitamins and/or the amide thereof. The stabilizing agent preferably comprises nicotinic acid, which is also referred to as vitamin B3, pyridine-3-carboxylic acid or niacin, and/or the amide thereof, nicotinamide, which is also referred to as niacinamide, nicotinic acid amide or pyridine-3-carboxamide. The concentration of the at least one stabilizing agent, in particular of the at least one antioxidant, can be between 0.01 mg/ml and 60 mg/ml, preferably between 1 mg/ml and 50 mg/ml, most preferably between 5 mg/ml and 30 mg/ml.

To simplify the method according to the invention, it can be provided for the at least one stabilizing agent to be added together with the acid solution. Preferably, the at least one stabilizing agent is dissolved in the acid solution and in particular has a concentration between 0.05 N and 1.5 N, in particular between 0.1 N and 1 N, most preferably between 0.25 N and 1 N. The acid solution, in particular with the at least one stabilizing agent dissolved in the acid solution, is preferably added at a cooler temperature than the ambient temperature, in particular at a temperature between 1° C. and 12° C., preferably between 4° C. and 8° C. It has been found that an acid solution that is cooler than the ambient temperature improves the stated effect on the molecules of the hyaluronic acid and/or the salt thereof.

Preferably, it is provided for the composition to be sterilized, in particular using heat, for example moist heat. In particular, autoclaving of the composition can be provided for this purpose. The composition can be sterilized at a temperature above 100° C., preferably above 110° C., in particular above 120° C. In addition, the duration of the sterilization can be between 1 s and 30 min. The sterilization can have at least one sterilization cycle, wherein the sterilization cycle can, for example, provide sterilization at 121° C. for 20 minutes, at 126° C. for 7 minutes, or at 130° C. for 3 minutes.

The hyaluronic acid and/or the salt of hyaluronic acid of the composition according to the invention can have a molecular weight between 0.01 Da and 6 MDa, in particular between 0.1 Da and 3.5 MDa. In addition, the composition according to the invention can be in the form of a hydrogel.

The composition according to the invention can have a concentration of hyaluronic acid and/or the salt thereof between 0.01 mg/ml and 60 mg/ml, in particular between 5 mg/ml and 35 mg/ml, preferably between 10 mg/ml and 25 mg/ml. In addition, the composition according to the invention can have a concentration of the at least one biologically active molecule between 0.01 mg/ml and 50 mg/ml, in particular between 1 mg/ml and 30 mg/ml, preferably between 1 mg/ml and 20 mg/ml. The composition according to the invention can have a concentration of the at least one stabilizing agent between 1 mg/ml and 60 mg/ml, in particular between 5 mg/ml and 50 mg/ml, preferably between 10 mg/ml and 30 mg/ml. The stabilizing agent can in particular be diluted in an acidic solution and in particular have a concentration between 0.05 N and 1.5 N, in particular between 0.1 N and 1 N, preferably between 0.25 N and 1 N. The composition according to the invention can be injectable, in particular accommodated in a syringe and/or vial.

The composition can be used according to the invention for stimulating and/or promoting the regeneration of tissue, in particular skin tissue, and/or for moisturizing tissue and/or for protecting tissue and/or for stimulating hair growth. In addition, it can be used by intradermal and/or subcutaneous injection.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWING

The present invention will become more fully understood from the detailed description given hereinbelow and the accompanying drawing which are given by way of illustration only, and thus, are not limitive of the present invention, and wherein the sole FIGURE shows a table of concentration changes over time for compositions G1 and REF1.

### DETAILED DESCRIPTION

In the following, a method according to the invention for preparing a composition G1 and a method for preparing a reference composition REF1 are described.

To prepare the composition G1 according to the invention, 22 g of powdered sodium hyaluronate (NaHA), which corresponds to the sodium salt of hyaluronic acid, with a molecular weight of approximately 2.1 MDa and a moisture content of 9.7%, are first weighed and prepared. To this, 132 g of an aqueous solution of sodium hydroxide (NaOH) at a concentration of 0.25 N are then added as an alkaline solution. The powder dissolves within 1 hour under mechanical homogenization. Then 320 g of a phosphate solution containing L-cysteine, the naturally occurring enantiomer of cysteine, are added, wherein L-cysteine is diluted in a ratio of 1:15. The composition is then mechanically homogenized for 30 minutes. A phosphate buffer solution cooled to a temperature between 4° C. and 8° C. is then added so that the composition has a pH of 7.2, wherein mechanical homogenization then takes place. To this composition is added a phosphate solution containing 30 g of nicotinamide, which corresponds to the amide of nicotinic acid, wherein mechanical homogenization is then carried out for a duration of 2 hours. The resulting gel-like composition according to the invention is designated G1. The composition G1 according to the invention has a concentration of hyaluronic acid of 10 mg/g, a concentration of L-cysteine of 10 mg/g, and a concentration of 15 mg/g nicotinamide, in each case in relation to the total composition. The composition G1 according to the invention is placed in a glass vial made of borosilicate glass and autoclaved at a temperature of 121° C. for 20 min.

To prepare the reference composition REF1, 22 g of powdered sodium hyaluronate (NaHA) with a molecular weight of approximately 2.1 MDa and a moisture content of 9.7% are weighed and prepared. To this are added 20 g L-cysteine and 30 g nicotinamide. A phosphate buffer solution is then added and the composition is then mechanically homogenized for a duration of 3.5 hours. The gel-like reference composition then obtained is designated REF1 and has a concentration of hyaluronic acid of 10 mg/g, a concentration of L-cysteine of 10 mg/g, and a concentration of nicotinamide of 15 mg/g, again each in relation to the total composition. The reference composition REF1 is placed in a borosilicate glass vial and autoclaved at a temperature of 121° C. for 20 min.

The concentrations of hyaluronic acid, L-cysteine and nicotinamide of the composition G1 prepared according to the invention correspond to the corresponding concentrations of the reference composition REF1, wherein the composition G1 according to the invention differs from the reference composition REF1 by the preparation method thereof.

In the following, the release dynamics with respect to L-cysteine and nicotinamide are investigated according to the FIGURE, on the one hand for the composition G1 prepared according to the invention and on the other hand for the reference composition REF1. It will be shown that the release dynamics of the two compositions G1 and REF1 differ, which is primarily attributable to the different preparation methods.

5 g of the composition G1 prepared according to the invention and 5 g of the reference composition REF1 are each placed in a small dialysis bag in 20 g of a saline or NaCl solution, with dialysis being carried out at different time intervals. After the durations indicated with “t” in the first row of the FIGURE, the dialysis is stopped, the compositions G1 and REF1 each being homogenized with a spatula, and the concentrations of L-cysteine and nicotinamide each being determined by UV spectroscopy. The concentrations thus determined for the compositions G1 and REF1 are shown in the rows of the FIGURE, wherein the concentrations each correspond to the remaining proportion of the components mentioned in the FIGURE in relation to the original concentration of the components before the start of dialysis. The concentration data in the FIGURE are therefore to be understood as relative concentrations. The concentrations are each determined as weight percent, % (w/w).

The first row of the FIGURE shows the different times at which dialysis was performed. The second row shows the concentrations of L-cysteine (“L-cysteine”) of the composition G1 prepared by the method according to the invention determined at the times mentioned in the first row, while the third row shows the concentrations of L-cysteine of the reference composition REF1. The fourth row of the FIGURE shows the concentrations of nicotinamide (“niacinamide”) at the times mentioned for the composition G1 prepared according to the invention, and the fifth row shows the concentrations of nicotinamide for the reference composition REF1.

It should be noted that for both compositions G1 and REF1 an almost complete release of both L-cysteine and nicotinamide occurs after 8 h at the latest. However, there are significant differences in the release dynamics between compositions G1 and REF1, which are associated with a significant advantage in clinical use for the composition G1 prepared according to the invention.

If the second row is compared with the third row according to the FIGURE, it can be seen that after a time of 1.5 h, the composition G1 still has an L-cysteine concentration of 0.63, i.e. 63%, while the L-cysteine concentration of the reference composition REF1 has fallen to 0.25 at the same time, i.e. by more than half compared to the composition G1. After 4 h, the composition G1 has an L-cysteine concentration of 0.38, which is higher by a factor of 4 than the L-cysteine concentration of 0.08 of the reference composition REF1 at the same time point. In the composition G1 prepared according to the invention, the release of L-cysteine as a biologically active molecule is therefore significantly delayed compared to the reference composition REF1. One explanation for this is that the alkaline solution initially causes the hyaluronic acid chains to expand, after which the cooled hydrochloric acid added after the addition of L-cysteine as a biologically active molecule to neutralize the composition causes a steric hindrance of the hyaluronic acid molecules in such a way that they are contracted or compressed by the hydrochloric acid and thus enclose the L-cysteine molecules between them for longer. The result is the sustained release of L-cysteine.

With regard to nicotinamide, the concentration progressions in the FIGURE show that the nicotinamide concentrations had fallen to almost 0 in both compositions G1 and REF1 after 4 h; almost all of the nicotinamide had been released by this time. In comparison, it is noticeable that after 1.5 h the nicotinamide concentration of the composition G1 fell faster, with a value of 0.17, than in the case of the reference composition REF1, which has a nicotinamide concentration of 0.23 after 1.5 h. It is possible that the steric hindrance in the case of nicotinamide is not significant or is only significant to a very small extent, since nicotinamide is only added at the end of the method of preparing the composition G1, in particular after the hyaluronic acid molecules have contracted, and therefore the nicotinamide molecules are not subject to any delayed release dynamics.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for preparing a composition containing hyaluronic acid and/or a salt of hyaluronic acid, and at least one biologically active molecule, the method comprising:
providing a solution that contains hyaluronic acid and/or a salt of hyaluronic acid;
adding an alkaline solution;
adding the at least one biologically active molecule; and
adding an acid solution to obtain the composition.

2. The method according to claim 1, wherein the alkaline solution contains water and a hydroxide component, in particular an alkali metal hydroxide.

3. The method according to claim 1, wherein the at least one biologically active molecule causes a biological effect on skin cells, in particular fibroblasts, keratinocytes, melanocytes and/or adipocytes.

4. The method according to claim 1, wherein the at least one biologically active molecule is selected from the group of amino acid, amino ester, hydroxy ester, aldehyde, a derivative of aldehyde, a ketone containing multiple hydroxyl groups, a derivative of a ketone containing multiple hydroxyl groups, cysteine, and/or L-cysteine.

5. The method according to claim 1, wherein the acid solution contains concentrated hydrochloric acid.

6. The method according to claim 1, wherein the method provides for the addition of at least one stabilizing agent.

7. The method according to claim 6, wherein the at least one stabilizing agent comprises at least one antioxidant, in particular an antioxidant vitamin.

8. The method according to claim 6, wherein the at least one stabilizing agent is added together with the acid solution.

9. The method according to claim 8, wherein the at least one stabilizing agent is dissolved in the acid solution and in particular has a concentration between 0.05 N and 1.5 N, between 0.1 N and 1 N, or between 0.25 N and 1 N.

10. The method according to claim 1, wherein the composition is sterilized.

11. A composition prepared by a method of claim 1.

12. The composition according to claim 11, wherein the composition is for aesthetic and/or therapeutic use in humans and/or animals.

13. The composition according to claim 12, wherein the composition is for stimulating and/or promoting the regeneration of tissue, in particular skin tissue, and/or for moisturizing tissue and/or for protecting tissue and/or for stimulating hair growth.

14. The composition according to claim 12, wherein the composition is carried out by intradermal and/or subcutaneous injection.
